(19) United States
(12) Patent Application Publication
(10) Pub. No.: US 2024/0417724 A1
(43) Pub. Date: Dec. 19, 2024

(54) AN ANTI-INFLAMMATORY COMPOSITION AND A METHOD OF OBTAINING THE SAME

(71) Applicant: VASTU VIHAR BIOTECH PRIVATE LIMITED, Bihar, Patna (IN)

(72) Inventor: Vinay KUMAR TIWARY, Bihar, Patna (IN)

(73) Assignee: VASTU VIHAR BIOTECH PRIVATE LIMITED, Bihar, Patna (IN)

(21) Appl. No.: 18/700,215

(22) PCT Filed: Dec. 2, 2021
(86) PCT No.: PCT/IB2021/061243

§ 371 Date: Apr. 10, 2024

(30) Foreign Application Priority Data
Oct. 11, 2021 (IN) 202131046340

(51) Int. Cl. C12N 15/113; A61K 9/50; A61P 11/00; A61P 15/08; A61P 17/00; A61P 25/28

(52) U.S. Cl. CPC C12N 15/113; A61K 9/5068; A61P 11/00; A61P 15/08; A61P 17/00; A61P 25/28; C12N 2310/14; C12N 2310/141

(57) ABSTRACT

The present invention relates to an anti-inflammatory composition and also a method of obtaining the same. The anti-inflammatory composition of the present invention is capable of downregulating the inflammatory genes and comprises an extracellular ribonucleic acid (exRNA) encapsulated in an extracellular vesicle (EV) and other suitable excipients wherein both the extracellular ribonucleic acid (exRNA) and extracellular vesicle (EV) are derived from extra polymeric substances (EPS) of gram-positive non-pathogenic bacteria.

### RELATED APPLICATION

This application claims the benefit to Indian Application No. IN202131046340, filed on Oct. 11, 2021, the contents of which are incorporated by reference herein.

### BACKGROUND OF THE INVENTION

Autoimmune disease can be broadly defined as specific adaptive immune response that is mounted against self-antigens. Adaptive immunity is acquired immunity or specific immunity present in vertebrates, specific to the pathogen presented to the body. The adaptive immune response is meant to attack non-self-pathogens but can get aberrant at times and attack self-antigens. Self-antigens, in relation to autoimmune diseases, are cellular proteins, peptides, enzyme complexes, ribonucleoprotein complexes, DNA, and post-translationally modified antigens against which autoantibodies are directed. Self-antigen are therefore antigens present in the body of an individual. The adaptive immunity is normally directed against a foreign antigen and results in clearance of the antigen from the body. The cytotoxic T cells in the body are responsible for destruction and clearance of, for example virus-infected cells, through cell-mediated immunity. On the other hand the soluble antigens are cleared, by antibody-mediated immunity (AMI), by formation of immune complexes of antibody and antigen, which are taken up by cells of the mononuclear phagocytic system such as macrophages. The adaptive immune response that develops against self-antigens present in the body of an individual are sustained immune response that occurs against the self-antigen, as it is impossible for immune effector mechanisms to eliminate the antigen completely from the body. The consequence is that the effector pathways of immunity cause chronic inflammatory injury to tissues, which may prove lethal. The mechanisms of tissue damage in autoimmune diseases are essentially the same as in the protective immunity and in hypersensitivity diseases.

Psoriasis is a chronic inflammatory dermatosis. The aetiology of psoriasis involves genetic, metabolic, and immunologic mechanisms. T-helper type-1 (Th1 type) of immune response, inflammatory cells, elevated TNF-α and other cytokines, chemokines, and growth factors are mainstay of inflammatory response in the dermal microenvironment in Psoriasis. The epidermal hyper proliferation occurs secondarily. Psoriasis, has been viewed as an autoimmune disease as the flare-ups in psoriasis can be triggered by bacterial microbiota by molecular mimicry. Homologous peptides between streptococcal and keratin proteins, can cause CD8+ T cells' response to these homologous peptides. Pathways associated with autoimmunity such as T-cell development in thymus or periphery, T-cell proliferation, or activation that may include immune synapse formation regulators have been demonstrated in psoriasis.

Psoriasis has a complex pathophysiology triggered by excessive proliferation of keratinocytes in which the debris i.e. Pathogen-associated molecular patterns (PAMPs) of the bacteria play a crucial role. (https://doi.org/10.1128/CMR.00046-08). PAMPs are molecules with conserved motifs that are associated with bacterial infection that serve as ligands for host pattern recognition molecules such as Toll-like receptors. PAMPs leads to activation of Toll-like receptors. Thus in the process, TLR9 is activated and further leads to the activation of pro-inflammatory cytokines. The active crated plasmacytoid dendritic cells secrete IFNγ, i.e. Interferon-alfa, which further activates proinflammatory cytokines namely; IL-1β, IL-6, IFNγ, and TNFα, along with myeloid dendritic cells. These dendritic cells are responsible for the production of IL-12 and IL-23, which ultimately leads to the activation of TH1 and TH17 and certain cytokines such as TNFα, IL-17 and IL-22. 3394; (https://doi.org/10.3390/ijms20143394). The activation of all these cytokines, and chemokines altogether result in increased inflammation.

Two major classes of non-coding RNA; long non-coding RNA (lncRNA) and microRNA (miRNA) are also found to play a role in the pathogenesis of psoriasis (https://doi.org/10.1128/CMR.00046-08). Various studies have shown that psoriasis patients have significantly higher levels of miRNA expression as compared to healthy individuals. For example, the upregulated miR-31, (DOI: 10.4049/jimmunol.1202695) has been found to be associated with the regulation of NF-KB pathway as well as the endothelial cell-activating signals produced by keratinocytes. Similarly, lncRNA, that are said to play a significant role in epigenetic silencing, apoptosis, splicing regulation, translation control, and cell cycle regulation are also associated with the regulation of variety of skin pathologies, including psoriasis, wound healing as well as skin cancer. Standard therapies often include topical corticosteroids, vitamin D analogues; retinoids; calcineurin inhibitors, salicylic acid, coal tar etc. Light therapy is a also used as first-line treatment for moderate to severe psoriasis. Moderate to severe psoriasis may be treated with oral or injected steroids, retinoids or methotrexate and biologics (https://www.mayoclinic.org/diseases-conditions/psoriasis/diagnosis-treatment/drc-20355845).

Atopic dermatitis (AD), is an inflammatory skin diseases caused due to a disturbed skin barrier as well as aberrant immune reactions against harmless allergens. Serum total IgE levels are often drastically increased in atopic patients, but the specificity of most of the total IgE remains unclear. It has been concluded that AD is indeed associated with IgE-autoreactivity. The pathogenesis of AD depends on many factors such as genetic factors, defective skin barrier, immune disbalance/abnormalities and environmental factors. It is generally thought that the elevated IgE responses and also the eosinophilia observed in the majority of patients with AD reflects the increased expression of TH2 cytokines, that is, increased IL-4, IL-5, IL-9, IL-10, IL-13, IL-17, IL-23 and IL-33 with a concomitant increase in IFN-γ expression. In this regard, peripheral blood lymphocytes from patients with AD have been reported to secrete increased amounts of IL-4 and express abnormally high levels of IL-4 receptor as well as increased IL-13. AD have also been found to have an increased capacity to produce IFN-γ in response to a number of stimuli. There have also been a number of studies demonstrating increased frequency of allergen-specific T cells producing increased IL-4, IL-5, IFN-γ and TGF-β in the peripheral blood and skin lesions of patients with AD. Upon stimulation with allergens, skin barrier derived TGF-β and CTLA4 stimulates DCs to derive Th2 responses. (https://dx.doi.org/10.4172%2F2155-9899.1000110) (https://doi.org/10.3390/ijms21041314). Standard of care include Topical corticosteroids (TCS), Topical calcineurin inhibitors (TCI), Topical antimicrobials, antiseptics and Phototherapy Alzheimer's disease is a progressive disease that destroys memory and other important mental functions. Brain cell connections and the cells themselves degenerate and die, eventually destroying memory and other important mental functions. The hippocampus is a key region in neurological diseases specially in Alzheimer's disease. Damage to the hippocampus region causes cognitive dysfunction that leads to Alzheimer's disease. In Alzheimer's altered amyloid precursor processing generates R amyloid peptide, which accumulates in the brain in Alzheimer's disease. The beta-amyloid deposition and neurofibrillary tangles lead to loss of synapses and neurons, which results in gross atrophy of the affected areas of the brain, typically starting at the mesial temporal lobe. Neurons may break down and many brain regions to shrink. By the final stages of Alzheimer's this process called atrophy is widespread causing significant loss of brain volume and is caused by the abnormal build-up of proteins called amyloid deposits which form plaques around brain cells.

Some of the key inflammatory cytokines involved in pathogenesis of Alzheimer's' are Presenilin 1, cathepsin B, tumour necrosis factor (TNF-α), Interleukin 1 beta, Interleukin 6. Presenilin 1 (PSEN 1) genes encode the major component of γ-secretase, which is responsible for sequential proteolytic cleavages of amyloid precursor proteins and the subsequent formation of amyloid § peptides. The cathepsin B is involved in the degradation of A3 and in the production of A3 Peptides. Inflammatory cytokines including tumour necrosis factor (TNF a), Interleukin 1 beta, Interleukin 6, suspend phagocytosis of amyloid A3 in patients with Alzheimer's disease. (https://doi.org/10.1038/s41598-018-30487-6); (https://doi.org/10.1371/journal.pmed.1002270); (DOI: 10.3233/JAD-2010-101023).

Cholinesterase inhibitors have been the cornerstone of treatment for patients with Alzheimer's disease and are recommended by the American Academy of Neurology practice parameter as standard of care for treatment of patients with this condition (https://www.ncbi.nlm.nih.gov/pmc/articles/PMC1896294/).

Polycystic ovary syndrome (PCOS) is a complex condition characterized by elevated androgen levels, menstrual irregularities, and/or small cysts on one or both ovaries. The pathophysiology of PCOS involves primary defects in the hypothalamic-pituitary axis, insulin secretion and action, and ovarian function. PCOS has been linked to insulin resistance and obesity. The insulin helps to regulate ovarian function, and the ovaries respond to excess insulin by producing androgens, which can lead to anovulation. Follicular maturation arrest is a hallmark sign that indicates the existence of ovarian abnormality.

These cytokines involved in the pathophysiology of PCOD involve overexpression of TNF-alpha, IL6, NFkB, and downregulation of cxcl-10, Il-12, IL-15, and IL-18. Various genes overexpressed in PCOD may include ESR-1, KISS-1. Inflammatory cytokines like IL-6, IL-12, IL-15, IL-18, NFKB. Similarly, KISS-1 is a neuropeptide that positively regulates gonadotropin releasing hormone kisspeptin, responsible for the higher serum LH level in PCOS patients. (https://doi.org/10.3109/09513590.2012.760195), (https://doi.org/10.1186/s12929-018-0452-2). The standard of care in PCOS include hormonal contraceptives, progestins and metformin (https://www.ncbi.nlm.nih.gov/books/NBK278959/).

Chronic obstructive pulmonary disease (COPD) is characterized by persistent airflow limitation and alveolar parenchymal cells depletion. It has a very high incidence in aging populations. Role of C-reactive protein (CRP), sputum or blood eosinophils, IL-1 β, caspase-1, IL-18, CXCL10, IL-8CXCL8 and MCP-1 have been implicated in COPD.

Recently many studies investigated the role of NLR (Nod-like receptors) family in COPD and the NLRP-3 (NLR containing a Pyrin domain 3). Briefly, the activation of NLRP-3 complex leads to the activation of Caspase-1 which causes the cleavage and maturation of the pro-inflammatory cytokines IL-1β and IL-18. Likewise, the signaling pathway of NLRP-3 is found to be a major mediator of immune response to exposure of cigarette smoke and airborne insults, through activation of toll-like receptors on epithelial cells and activation of immunological cells such as neutrophils and dendritic cells. Inhaled irritants activate epithelial cells and alveolar macrophages, which play a central role in type 1 airway inflammation by releasing cytokines and chemokines. Alveolar macrophages and neutrophils release proteases, such as MMPs and neutrophil elastase, which cause elastin degradation and further results in alveolar wall destruction. Dendritic cells are an important link between innate immunity and adaptive immunity, locating near the epithelium surface to sense the entry of inhaled irritants. IL8, IL6, IL22, IL17A, interleukins; ROS, reactive oxygen species; DAMPs, damage-associated molecular patterns; PAMPs, pathogen-associated molecular patterns; TNF-α, tumor necrosis factor-α; TGF-β1, transforming growth factor-β1; CCL2, C-C motif chemokine; CXCL1, CXCL8, CXCL9, CXCL10, CXCL11 chemokine (C-X-C motif) ligand; NK cell, natural killer cell; ILC3, innate lymphoid cell 3; Th, helper T lymphocyte; Tc, cytotoxic T lymphocyte; G-CSF, granulocyte colony-stimulating factor; IFN-γ, interferon-gamma; MMP28s, matrix metalloproteinases all these are found to be involved in pathogenesis of COPD. (https://doi.org/10.1371/journal.pone.0214622_9:759-773; pmid:25125975; 9(6): 434-440.9(1); e85243. pmid:24416369, 184: 662-671. pmid:21680942), Standard of care include anticholinergic, beta2 agonist and Inhaled corticosteroids (https://www.aafp.org/afp/2013/1115/p655.html).

Thus the role of the inflammatory cytokines are clear in the above mentioned autoimmune diseases. The standard of care for each condition although give symptomatic relief to the patient but patient compliance is often difficult due to long-term treatment modalities and sometimes due to high cost of the therapy. Often the standard of care is mostly directed towards suppressing the immune response rather than neutralizing or controlling the hyper immune response, therefore a need for a better drug that can fulfil this lacuna still persists.

Few of the prior arts are aimed at the inflammatory response generated in the autoimmune diseases.

US20170224617A1 published on 10 August 2017, disclose a compositions containing a nanolipogel for sustained delivery of an effective amount of one or more active agents such as a drug for treating an inflammatory or autoimmune disease or disorder. The nanolipogel includes a lipid bilayer surrounding a hydrogel core, which may optionally include a host molecule, for example, an absorbent such as a cyclodextrin or ion-exchange resin. The active agents can be an immunosuppressant. The nanolipogel includes a targeting moiety that increases specificity of the particle for activated T cells or antigen presenting cells. however, this prior art is directed towards the delivery of the drug to target and not at an anti-inflammatory drug.

US 2011/0081320A1 published on 7 April 2011 disclose a method administering a source of a protease that destroys or deactivates an immunogen, mimic and/or antigen specific to a particular autoimmune disease before it encounters the immune system. The method therefore is aimed at clearance of the antigen from the immune system rather than being directed against the hyper immune response in autoimmune diseases.

The prior art therefore discloses some of the compositions that can mitigate the anti-inflammatory response in the autoimmune diseases. however there is still a need to develop effective anti-inflammatory composition that can be scaled up to the industrial level with ease at an effective cost and have better and more effective mechanism for controlling/suppressing the hyper inflammatory response that are the main stay of the autoimmune diseases.

### OBJECT OF THE INVENTION

The object of the present invention is to develop anti-inflammatory composition that downregulates the inflammatory cytokines and transcription factors involved in the inflammatory pathways in autoimmune diseases.

Another object of the present invention is to develop composition for treatment of psoriasis, asthma, atopic dermatitis, Alzheimer's, Polycystic ovarian disorder (PCOD), Coronary obstructive pulmonary disorder (COPD) and other autoimmune disease that are based on hyperactive inflammatory response.

Yet another object of the present invention is to develop a method of conversion of biofilm-associated extra polymeric substances (EPS) into vesicular compartment (extracellular vesicles) containing extracellular ribonucleic acid (exRNA) that suppress the inflammatory genes in the autoimmune diseases.

### SUMMARY OF THE INVENTION

This summary is provided to introduce a selection of concepts in a simplified format that is further described in the detailed description of the invention. This summary is neither intended to identify key or essential inventive concepts of the invention and nor is it intended for determining the scope of the invention.

The present invention discloses an oral and topical anti-inflammatory compositions that down regulates inflammatory genes comprising; extracellular ribonucleic acid (exRNA); extracellular vesicle bound by a single membrane phospholipid layer comprising glycopeptides; lipopeptide; polysaccharides; and small peptides; phosphate buffer saline; sodium chloride; glycerin/ethanol; and double distilled water, wherein the extracellular ribonucleic acid (exRNA) is encapsulated in extracellular vesicle (EV), wherein the extracellular ribonucleic acid (exRNA) and extracellular vesicle (EV) are derived from extra polymeric substances (EPS).

The present invention also discloses a method of conversion of biofilm-associated extra polymeric substances (EPS) into vesicular compartment (extracellular vesicles) comprising suspension of the extra polymeric (EPS) substances in conversion buffer to obtain EPS suspension; homogenization of the EPS suspension followed by the centrifugation of the homogenized EPS suspension to obtain a EPS supernatant; and filtration of the EPS supernatant to obtain extracellular vesicles wherein the extracellular vesicles encapsulates extracellular ribonucleic acid (exRNA), wherein the suspension is made is 1:20 in conversion buffer.

The present invention also discloses a kit constituting components comprising extracellular ribonucleic acid (exRNA) 4-12 μg/ml; phosphate buffer saline 0.4-1%, pH −7.4; sodium chloride 0.9%; glycerin 1-5%; pre-filled syringe containing double-distilled water; spray bottle for topical application; and instruction manual, wherein the extracellular ribonucleic acid (exRNA) is encapsulated in extracellular vesicle (EV), wherein the components i-v are provided as lyophilized powder in a glass vial and are to be reconstituted with the double-distilled water provided in the pre-filled syringe, wherein the instructions manual have necessary instruction for reconstitution of the components contained in the glass vial to obtain a ready-to-use topical anti-inflammatory composition.

The present invention discloses a kit constituting components comprising extracellular ribonucleic acid (exRNA) 4-12 μg/ml; phosphate buffer saline 0.4-1%, pH −7.4; sodium chloride 0.9%; ethanol in the range of 10-20%; pre-filled containing double-distilled water syringe; and instruction manual, wherein the extracellular ribonucleic acid (exRNA) is encapsulated in extracellular vesicle (EV), wherein the components i-v are provided as lyophilized powder in a glass vial and are to be reconstituted with the double-distilled water provided in the pre-filled syringe, wherein the instructions manual have necessary instruction for reconstitution of the components contained in the glass vial to obtain a ready-to-use oral anti-inflammatory composition.

### BRIEF DESCRIPTION OF FIGURES

The above mentioned objectives and descriptions, features, aspects, and advantages of the present invention will become better understood when the following detailed description is read with reference to the accompanying figures in which like characters represent like parts throughout the figures, wherein:

FIG. 1 depicts m-RNA level expression in IL-17, IL-23, IL-6, NF-KB, TNF-α, TGF-β, IFN-γ, TLR-9 in healthy rats, Control (diseased induced group-psoriasis) and the Treatment group-(diseased induced-treated with M9 composition group) in psoriasis model;

FIG. 2 A-C depicts M9 composition treated mice (left) and Control (diseased induced group-psoriasis; The figure clearly shows psoriatic lesions in the Control group and normal skin morphology after 6-8^{th} days treatment with M9 composition in the Treatment group in psoriasis model;

FIG. 3A depicts the amplification curve through RT PCR and FIG. 3B shows levels of mRNA of interleukins measured using real-time PCR in psoriasis model;

FIG. 4 depicts dermatitis in comparison of Control group and M-9 composition treatment and the recovery of the model rat from Atopic dermatitis in the M-9 composition treated group;

FIG. 5A depicts plot of observed lesion score of the model rat from day 1 to the last day of the treatment. Overall score was given according to the lesions appeared in the rat skin; FIG. 5B depicts comparisons of serum levels of pro-inflammatory cytokines IL-9, IL-10, IL-17, IL-23, IL-33 and IFN-gamma in all the three groups of rat model in atopic dermatitis; FIG. 5C depicts comparisons of serum levels of pro-inflammatory chemokines CCL-2, CCL-11, CCL-17, TGF-beta, CTLA4 and serine protease released by the skin cells KLK7 gamma in all the three groups of rat model in atopic dermatitis FIG. 6 depicts before (a) after (b) treatment with M9-1 composition in rat model in Alzheimer's;

FIG. 7 depicts mRNA levels of biomarkers and inflammatory cytokines which are downregulated after treatment of M9-1 composition in all the three groups of rat model in Alzheimer's;

FIG. 8 depicts downregulation of genes IL-17, IL-6, TGF-β, TNF-α in all the three groups of rat model in Chronic obstructive pulmonary disease (COPD) disease model; and

FIG. 9 depicts downregulation of genes IL-17, IL-6, TGF-β, TNF-α in all the three groups of rat model in Polycystic ovary syndrome (PCOS) disease model;

FIG. 10 depicts the exRNA isolation and gel electrophoresis of the same;

FIG. 11 depicts gram staining characteristic of the source bacteria in a 12 hours culture plate, used for obtaining EPS;

FIG. 12 depicts gram staining characteristic of the source bacteria in 18 hours culture plate, used for obtaining EPS;

FIG. 13 depicts gram staining characteristic of the source bacteria, in 30 hours culture plate used for obtaining EPS;

FIG. 14 depicts gram staining characteristic and OD of the source bacteria in a 36 hours culture plate used for obtaining EPS;

FIG. 15 depicts 280/260 ratio in the drug sample (1) blue line graph-sample 1 of the drug sample, (2) green line graph-sample 2 of the drug sample; (3) red line graph-control;

FIG. 16 depicts quantification of exRNA using a fluorophore;

FIG. 17 depicts mass to charge ratio as characterized in extracellular vesicle;

FIG. 18 depicts fingerprint of the composition on spectrophotometer at 230, 260, 280, 320 nm; and

FIG. 19 depicts size distribution by intensity reports of extravesicular revealed to be an anionic molecule with a zeta potential (−1) to (−30) mV and 20-500 nm.

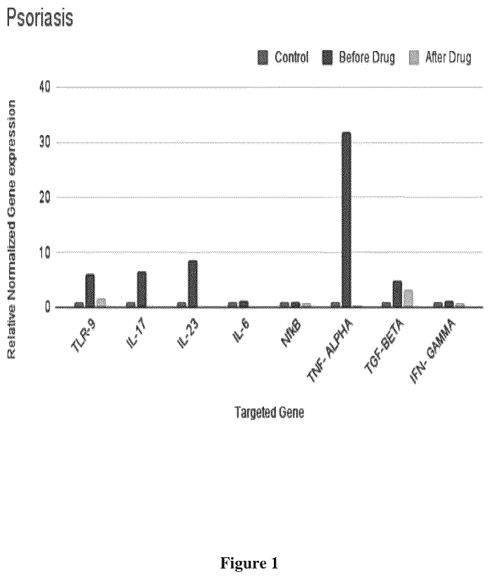

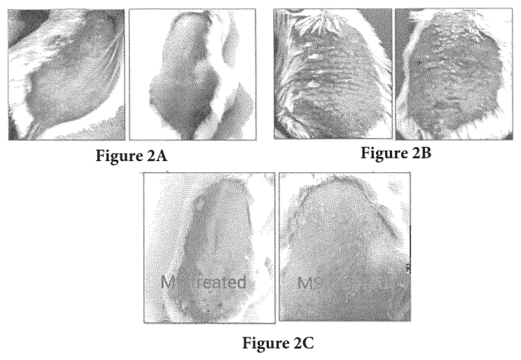

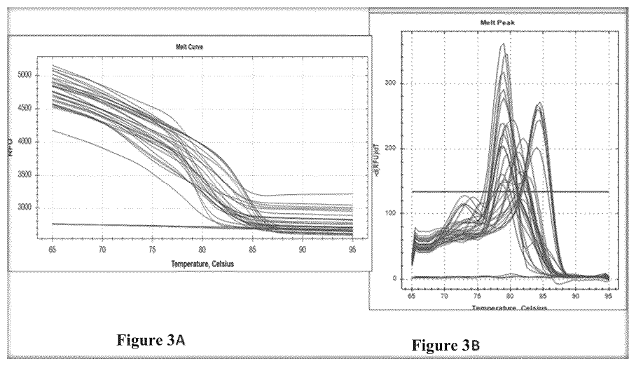

Further, skilled artisans will appreciate that elements in the figures are illustrated for simplicity and may not have been necessarily drawn to scale. For example, the flow charts illustrate the method in terms of the most prominent steps involved to help to improve understanding of the aspects of the present invention. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having benefit of the description herein.

### DETAILED DESCRIPTION

For the purpose of promoting an understanding of the principles of the invention, reference will now be made to the embodiment illustrated in the figures and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention.

Thus, appearances of the phrase “in an embodiment”, “in another embodiment” and similar language throughout this specification may, but do not necessarily, all refer to the same embodiment The terms “comprises”, “comprising”, or any other variations thereof, are intended to cover a non-exclusive inclusion, such that a process or method that comprises a list of steps does not include only those steps but may include other steps not expressly listed or inherent to such process or method.

Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components. Unless otherwise defined, all technical and scientific terms used herein have the same meaning as commonly understood by one of ordinary skill in the art to which this invention belongs. The system, methods, and examples provided herein are illustrative only and not intended to be limiting.

Extracellular vesicles (EVs) are derived from extra polymeric substances (EPS) of the non-pathogenic, gram-positive bacterial species. EVs are the lipid bound vesicles that consists of lipids (phospholipids), nucleic acids, lipopeptides, lipoteichoic acid, aspartate, alanine, aspartic acid, tryptophan, tyrosine, histidine, glutathione.

Extracellular RNA (exRNA) are RNA molecules which are encapsulated in extracellular vesicles derived from gram positive bacteria more specifically non-pathogenic bacteria.

exRNA may be selected from group of micro-RNA, siRNA, circular RNA, piwi RNA, gRNA.

Extracellular vesicles (EVs) are the lipid bound vesicles of the extracellular matrix (ECM) secreted out of the cell. The content or cargo of EV consists of lipids (mainly phospholipids), nucleic acids, lipopeptides, lipoteichoic acid, aspartate, alanine, arginine, aspartic acid, polysaccharides, propanoic acid, glutathione proteins, specifically proteins associated with the plasma membrane, cytosol and those involved in the lipid metabolism. Extracellular vesicles (EVs) are important players in autoimmune diseases, both in disease pathogenesis and as potential treatments. EVs can transport autoimmune triggers throughout the body, facilitating the process of antigen presentation. Thus there exist a link between cellular stress and EV biogenesis and intercellular trafficking in autoimmune diseases. In addition, EVs can also be effective treatments for autoimmune diseases. The diversity of cell types that produce EVs leads to a wide range of molecules to be present in EVs, and thus EVs have a wide range of physiological effects. EVs derived from dendritic cells, mesenchymal stem cells and some bacterial EVs have been shown to reduce inflammation. Since many autoimmune treatments are focused only on symptom management, EVs present a promising avenue for potential treatments. Bacterial pathogens quickly respond to changes in the environment to survive and propagate. Pathogenicity of a bacterium is a measure of virulence, which is manifested by the secretion of bacterial virulence factors via membrane blebs for invasion. The secretory vehicles called extracellular vesicles (EVs) are nanoparticles produced by most of the bacteria which have diverse biological functions and broad applications in immunology and biotechnology. The formation of vesicles appears to be a conserved process in both pathogenic and non-pathogenic bacteria. During the past few years, EVs have gained attention in all domains of life. EV secretion is now considered as a primordial feature of all living cells. An EV is defined as a spherical, membranous vesicle generated from a microbial cell surface with size ranging from 20 nm to 500 nm in diameter. Not only do they differ in size, but they also vary in morphology, composition, and biogenesis. Different terms are used for EVs in different organisms such as OMVs (outer membrane vesicles) in gram-negative bacteria and EVs or MVs (extracellular vesicles or membrane vesicles) in gram-positive bacteria.

In the 1960s, bacterial EVs were first reported in Escherichia coli, but their existence in gram-positive bacteria gained attention recently. In 1990, Dorward and Garon provided the first evidence of vesiculation in gram-positive bacteria. The release of spherical particles occurs by budding in the surrounding environment from the cells. It has been observed in several bacterial species belonging to gram-positive phyla Firmicutes and Actinobacteria. Bacterial membrane vesicles are widely accepted as bacterial secretion system known as membrane vesicles. EVs are produced by both gram positive and gram-negative bacteria. These are the important mediators to facilitate intracellular communications without cell-to-cell contact

The present invention discloses an oral and topical anti-inflammatory compositions that down regulates inflammatory genes comprising; extracellular ribonucleic acid (exRNA); extracellular vesicle bound by a single membrane phospholipid layer comprising glycopeptides; lipopeptide; polysaccharides; short chain fatty acids including propionic acid and small peptides; phosphate buffer saline; sodium chloride; glycerin; ethanol; and double distilled water, wherein the extracellular ribonucleic acid (exRNA) is encapsulated in extracellular vesicle (EV), wherein the extracellular ribonucleic acid (exRNA) and extracellular vesicle (EV) are derived from extra polymeric substances (EPS).

In one embodiment, the present application discloses a composition that may comprise extracellular ribonucleic acid (exRNA) in the range of 4-12 μg/ml; phosphate buffer saline in the range of 0.4-1%, pH −7.4; sodium chloride 0.9%; and glycerin in the range of 1-5%, wherein the extracellular ribonucleic acid (exRNA) is encapsulated in extracellular vesicle (EV).

In another embodiment, the extracellular vesicle may comprise single membrane phospholipid layer; lipopeptide; small peptides; and polysaccharides wherein the extracellular vesicle encapsulates extracellular ribonucleic acid (exRNA).

In yet another embodiment the extracellular vesicle may be an anionic molecule with a zeta potential (−1) to (−30) mV and 20-500 nm in size.

In one embodiment the extracellular ribonucleic acid (exRNA) inhibits the messenger-ribonucleic acid (m-RNA) of a particular gene) by antisense/ribozymal cleavage. The composition comprising the extra cellular vesicle encapsulating the exRNA acts by inhibiting the gene specific mRNA of the inflammatory genes or inhibiting synthesis of protein involved in the pathophysiology of the disease.

In another embodiment the extracellular ribonucleic acid (exRNA) inhibits the messenger-ribonucleic acid (m-RNA) of a particular gene which may be selected from a group comprising APOE (Apolipoprotein E), CTSB (Cathepsin-B), IL-1β (Interleukin-1 beta), IL-6 (Interleukin-6), Presenilin 1 (PSEN-1), TNF-α (Tumour necrosis factor-α), IL-4 (Interleukin-4), IL-5 (Interleukin-5), IL-6 (Interleukim-6), IL-9 (Interleukim-9), IL-12 (Interleukin-12), IL-13 (Interleukin-13), IL-15 (Interleukin-15), IL-17 (Interleukin-17), IL-18 (Interleukin-18), IL-23 (Interleukim-23), IL-33 (Interleukim-33) (Interleukim-6), TGF-β (Transforming growth factor-0), TNF-α (Tumour necrosis factor-α), IL-17, IL-23, IL-6, NfκB (Nuclear Factor kappa-light-chain-enhancer of activated B cells), TLR-9 (Toll like receptor-9), TNF-α, TGF-β, IFN-Υ (Interferon-γ), IRS-1 (Insulin receptor substrate 1), CXCL-10 (C-X-C motif chemokine ligand 10), ESR-1 (Estrogen Receptor 1), KISS-1, KLK-7 (Kallikrein-1), CCL-11 (C-C motif chemokine ligand-11), CCL-2 (C-C motif chemokine ligand-2) by antisense/ribozymal cleavage.

In yet another embodiment the extracellular ribonucleic acid (exRNA) may be selected from a group comprising micro Ribonucleic acid (miRNA), small interfering Ribonucleic acid (siRNA), guide Ribonucleic acid (gRNA) cRNA (circular Ribonucleic acid), piwi Ribonucleic acid (piRNA).

The exRNA represses the expression of mRNA targets by promoting translational repression and mRNA degradation. The repression be mediated by destruction of a target mRNA. exRNA may assemble with proteins to form a RNA-induced silencing complex (RISC) via Argonaute 2 (Ago 2). Formation of RISC precedes its binding to target mRNAs. RISC-exRNA complex within Ago2 identifies the target mRNA and a catalytic domain of Ago 2 cleaves the mRNA at a vulnerable phosphate of the mRNA. Cleaved mRNA cannot be translated and its encoded protein is not produced. RISC dissociates from its target, to repeat this process, a few RISC molecules thus can cleave many target mRNAs.

The present invention discloses an oral and topical anti-inflammatory compositions that down regulates inflammatory genes comprising; extracellular ribonucleic acid (exRNA); extracellular vesicle bound by a single membrane phospholipid layer comprising glycopeptides; lipopeptide; polysaccharides; short chain fatty acids including propionic acid and small peptides; phosphate buffer saline; sodium chloride; ethanol; and double distilled water, wherein the extracellular ribonucleic acid (exRNA) is encapsulated in extracellular vesicle (EV), wherein the extracellular ribonucleic acid (exRNA) and extracellular vesicle (EV) are derived from extra polymeric substances (EPS) that may be administered topically.

The present invention discloses an oral and topical anti-inflammatory compositions that down regulates inflammatory genes comprising; extracellular ribonucleic acid (exRNA); extracellular vesicle bound by a single membrane phospholipid layer comprising glycopeptides; lipopeptide; polysaccharides; short chain fatty acids including propionic acid; and small peptides; phosphate buffer saline; sodium chloride; glycerin; and double distilled water, wherein the extracellular ribonucleic acid (exRNA) is encapsulated in extracellular vesicle (EV), wherein the extracellular ribonucleic acid (exRNA) and extracellular vesicle (EV) are derived from extra polymeric substances (EPS) that may be administered orally.

In an embodiment, the anti-inflammatory composition may downregulate the pro-inflammatory gene selected from a group comprising APOE (Apolipoprotein E), CTSB (Cathepsin-B), IL-1 (Interleukin-1 beta), IL-6 (Interleukin-6), Presenilin 1 (PSEN-1), TNF-α (Tumour necrosis factor-α), IL-4 (Interleukin-4), IL-5 (Interleukin-5), IL-6 (Interleukim-6), IL-9 (Interleukim-9), IL-12 (Interleukin-12), IL-13 (Interleukin-13), IL-15 (Interleukin-15), IL-17 (Interleukin-17), IL-18 (Interleukin-18), IL-23 (Interleukim-23), IL-33 ((Interleukim-33) (Interleukim-6), TGF-β (Transforming growth factor-β), TNF-α (Tumour necrosis factor-α), IL-17, IL-23, IL-6, NfκB (Nuclear Factor kappa-light-chain-enhancer of activated B cells), TLR-9 (Toll like receptor-9), TNF-α, TGF-β, IFN-γ (Interferon-γ), IRS-1 (Insulin receptor substrate 1), CXCL-10 (C-X-C motif chemokine ligand 10), ESR-1 (Estrogen Receptor 1), KISS-1, KLK-7 (Kallikrein-1), CCL-11 (C-C motif chemokine ligand-11), CCL-2 (C-C motif chemokine ligand-2).

In an embodiment, the present application disclose a composition that may comprise an extracellular ribonucleic acid (exRNA) in the range of 4-12 μg/ml; phosphate buffer saline in the range of 0.4-1%, pH −7.4; sodium chloride 0.9%; and ethanol in the range of 10-20%.

In another embodiment composition of the present invention may be effective against autoimmune disease selected from psoriasis, asthma, atopic dermatitis, Alzheimer's, Polycystic ovarian disorder (PCOD), Coronary obstructive pulmonary disorder (COPD).

In still another embodiment the present invention disclose a method of conversion of biofilm-associated extra polymeric substances (EPS) into vesicular compartment (extracellular vesicles) comprising: suspension of the extra polymeric (EPS) substances in conversion buffer to obtain EPS suspension; homogenization of the EPS suspension followed by the centrifugation of the homogenized EPS suspension to obtain a EPS supernatant; and filtration of the EPS supernatant to obtain extracellular vesicles, wherein the extracellular vesicles encapsulates extracellular ribonucleic acid (exRNA), wherein the suspension is made is 1:20 in conversion buffer.

EVs from Gram-negative bacteria originate from the outer membrane, however in gram positive bacteria, it was believed for a long time that no EVs are secreted. EVs from Gram-negative bacteria are released from the outer membrane by a pinching-off process, encapsulating components from the periplasmic space as there is no physical barrier to the release of these EVs to the extracellular space. In contrast, in gram positive bacteria, mycobacteria and fungi due to presence of thick cell wall and lack an outer membrane, the mechanism of secretion of EVs were unclear. Although it is now believed that EVs may be secreted from the gram positive bacteria, to the best of our knowledge, this is the first ever report of a method of conversion of biofilm-associated extra polymeric substances (EPS) into vesicular compartment (extracellular vesicles).

The biofilm of a bacteria constituents a number of compounds such as peptide, lipoprotein, lipopeptide, peptidoglycan, periplasmic & cytoplasmic proteins, toxins and nucleic acids. The conversion buffer of the present application uses the lipoprotein, lipopeptide etc as surfactants present in the extra polymeric substances and results in formation of EV containing extracellular RNA (exRNA).

In one embodiment the extra polymeric substances (EPS) may be obtained from a non-pathogenic gram positive bacteria.

In another embodiment, the present invention discloses a conversion buffer that may be selected from a group comprising double distilled water; ethanol in the range of 0-30% in double distilled water; Dimethyl sulfoxide (DMSO) in the range of 0-5% in double distilled water; Sodium chloride (NaCl) (0.9% in double distilled water); Phosphate buffer saline (PBS) in the range of 0.4-1%, pH 7.4.

In yet another embodiment, the homogenization may be carried out at 1000 rpm for 1-5 minutes and the centrifugation may be carried out at 4500 rpm for 2-10 minutes. The filtration of the EPS supernatant may be carried out by twice with membrane filter pore size, 0.45 μm followed by membrane filter pore size, 0.22 μm.

In another embodiment the present invention disclose a method of treating autoimmune diseases including psoriasis, PCOD, COPD, asthma, Alzheimer's diseases, atopic dermatitis in an therapeutically effective amount.

In yet another embodiment the present invention discloses a kit constituting components comprising extracellular ribonucleic acid (exRNA) 4-12 μg/ml; phosphate buffer saline 0.4-1%, pH −7.4; sodium chloride 0.9%; glycerin 1-5%; pre-filled containing double-distilled water syringe; and instruction manual, wherein the extracellular ribonucleic acid (exRNA) is encapsulated in extracellular vesicle (EV), wherein the components i-v are provided as lyophilized powder in a glass vial, wherein the spray bottle is also provided for topical application and are to be reconstituted with the double-distilled water provided in the pre-filled syringe, wherein the instructions manual have necessary instruction for reconstitution of the components contained in the glass vial to obtain a ready-to-use topical anti-inflammatory composition.

An another embodiment of the present invention discloses kit constituting components comprising extracellular ribonucleic acid (exRNA) 4-12 μg/ml; phosphate buffer saline 0.4-1%, pH −7.4; sodium chloride 0.9%; ethanol in the range of 10-20%; pre-filled containing double-distilled water syringe; and instruction manual, wherein the extracellular ribonucleic acid (exRNA) is encapsulated in extracellular vesicle (EV), wherein the components i-v are provided as lyophilized powder in a glass vial and are to be reconstituted with the double-distilled water provided in the pre-filled syringe, wherein the instructions manual have necessary instruction for reconstitution of the components contained in the glass vial to obtain a ready-to-use oral anti-inflammatory composition.

### EXAMPLE

The following examples are for illustration purposes and are not to be construed as limiting the invention disclosed in this document to only the embodiments disclosed in these examples.

### Example 1

Gram-positive non-pathogenic bacteria was mutated in lab through homologous recombination method using a suitable bacteriophage strain, mutated bacteria was grown on a suitable culture media for 22-24 hrs. at 37° C. After formation of the full grown biofilm on the medium. The biofilm was scrapped from the surface and suspended in the conversion buffer to extract extra polymeric substances (EPS) from the biofilm. The conversion buffer used for suspension of extra polymeric substances (EPS) were carried out in 6 different solution-double distilled water; 10% ethanol (10 ml ethanol in double distilled water); 5% Dimethyl sulfoxide (DMSO) (5 ml DMSO in double distilled water); 0.9% Sodium chloride (NaCl) 0.9 gms NaCl dissolved in 90 ml double distilled water, making up the volume to 100 ml; Phosphate buffer saline (PBS), 0.819 gms PBS dissolved in 90 ml double distilled water, making up the volume to 100 ml, adjusting the pH to 7.4.

The homogenization of the mixture was carried out at room temperature (25° C.) for 1 minute in a homogenizer (NAMCO, Asia 1936). The suspension obtained after the homogenization is then centrifuged, (REMI-R8C) at 4500 rpm for 5 mins. The supernatant obtained in then filtered twice with membrane filter pore size, 0.45 μm followed by membrane filter pore size, 0.22 μm.

ExRNA Isolation from Extracellular Matrix:

For isolation of exRNA, 200 μl of 1:20 ratio of filtered sample (obtained above) was taken and 200 μl of isolation buffer was added, the solution was kept for 15 minutes at 56° C. and followed by addition of 300 μl of ethanol. The sample was loaded on to binding column and centrifuged at 6000 rpm for 1 min. The column was washed with 700 μl of wash buffer followed by centrifugation for 1 min, this was followed by a second wash with 500 μl of wash buffer followed by centrifugation for 1 minute. The unwanted material in the column gets washed and exRNA was then eluted and dried. Centrifugation done again for 3 mins at 6000 rpm followed by adding 50 μl of RNase free water for elution and solution was kept at 56° C. for 2 mins. Centrifugation was repeated at 12000 rpm for 1 minute to obtain exRNA.

Estimation of microRNA in the Drug Sample

Qubit™ RNA Assay Kits (Invitrogen) for RNA estimation in the drug sample obtained above. 190 μl of Qubit microRNA buffer and 1 μl of microRNA reagent was added and mixed properly. 20 μl of drug sample (1:20 ratio) was added. The sample kept for incubation at room temperature for 2 minutes. Finally, the reading of sample taken by using Qubit fluorophotometer. The OD was taken duplicates and the experiment was repeated thrice. The microRNA was estimated to be 4-12 μg/ml.

### Size Distribution of the Extracellular-Vesicular

The exosome is an anionic molecule with a zeta potential (−1) to (−30) mV and 20-500 nm in size as elucidated by the size distribution by intensity reports.

### Estimation of Total RNA in the Drug Sample by 260/280 Ratio

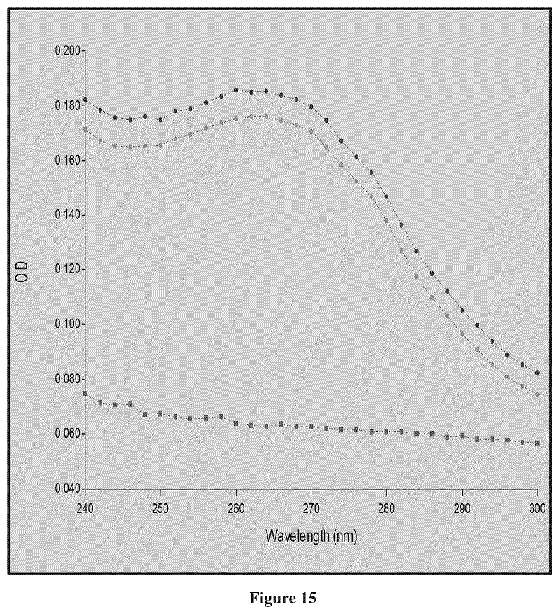

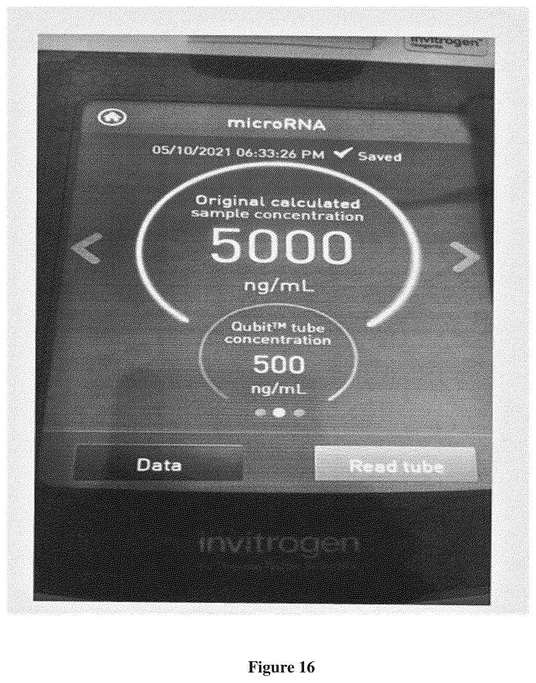

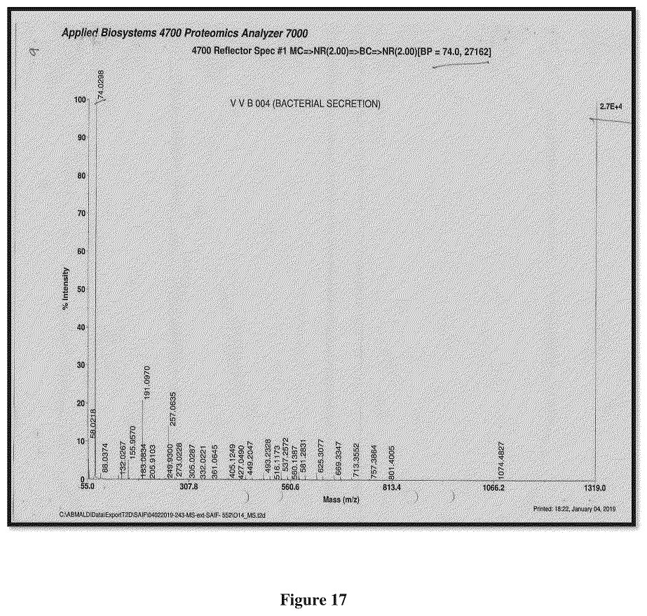

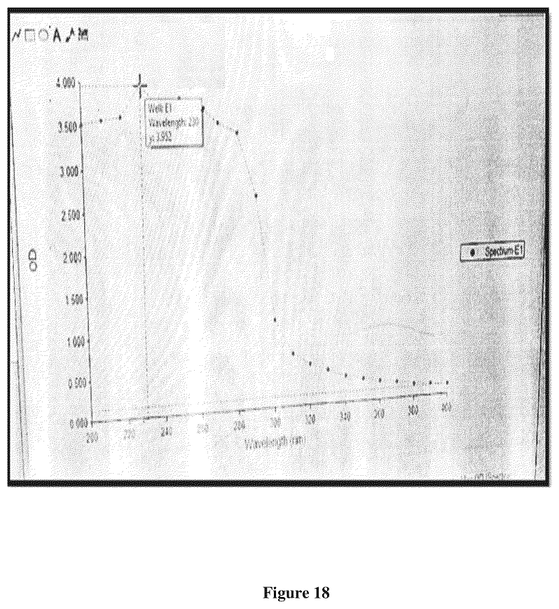

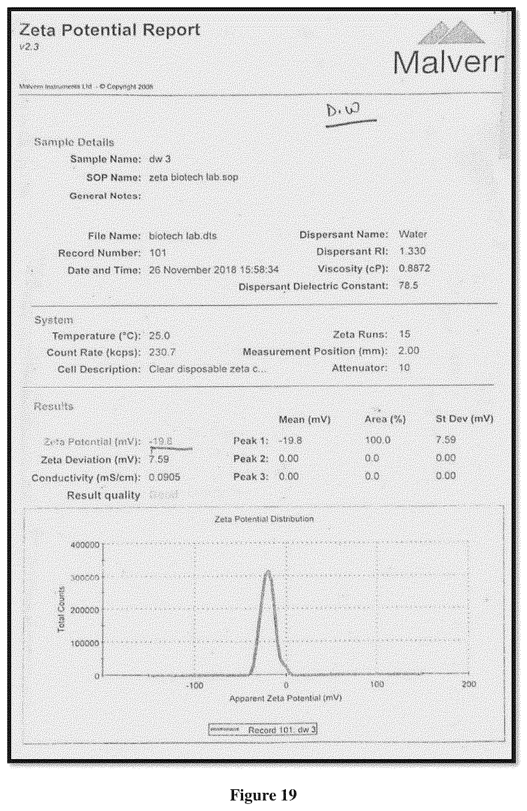

Drug sample was diluted in double distilled water in 1:20 ratio and homogenized. The homogenized sample was filtered by 0.22 μm Millipore membrane filter using a filter syringe. Samples were read at 240-300 nm using a BioTek Epoch 2 microplate spectrophotometer. The estimation of the 260/280 ratio was carried out. Nucleic acids have absorbance is maxima at 260 nm. The ratio of this absorbance maximum to the absorbance at 280 nm has been used as a measure of purity in both DNA and RNA extractions. A 260/280 ratio of ˜1.8 is generally accepted as “pure” for DNA; a ratio of ˜2.0 is generally accepted as “pure” for RNA. Samples showed 260/280 ratio in duplicate samples (sample 1 and 2) to be 1.926 and 1.899 respectively. The Total RNA was estimated to be 90.942 μg/ml and 92.856 μg/ml (Table 1; FIG. 15).

TABLE 1
Total RNA estimation in the drug sample

| | Reading at | Reading at | 260/280 | exRNA in |
|---|---|---|---|---|
| Sample | 260 nm | 280 nm | ratio | the sample |
| M9 Composition 1 | 0.114 | 0.059 | 1.926 | 90.942 ng/μL |
| (sample 1- blue | | | | or 90.942 μg/ml |
| line graph) | | | | |
| M9 Composition 1 | 0.116 | 0.061 | 1.899 | 92.856 ng/μL |
| (sample 2- green | | | | or 92.856 μg/ml |
| line graph) | | | | |

### Extracellular-Vesicular Layer

Exosomal layer was lysed with the help of a lysis buffer to dissolve the exosomal layer in the M9 and M9-1 (composition of the present invention (Sample A), and in a separate control no lysis buffer was added (Sample B). The sample A & B were passed by binding column. and reading (260/280 ratio) was taken in microtiter spectrophotometer. Nucleic acids have absorbance is maxima at 260 nm. The ratio of this absorbance maximum to the absorbance at 280 nm has been used as a measure of purity in both DNA and RNA extractions. A 260/280 ratio of ˜1.8 is generally accepted as “pure” for DNA; a ratio of ˜2.0 is generally accepted as “pure” for RNA. In Sample A, the (260/280) ratio of the reading obtained was 2.122 and the concentration of RNA was detected to be of 12.669 ng/μl. Whereas, in Sample B the (260/280), the ratio of the reading obtained was 1.417 (same as distilled water) and its concentration was 2.695 ng/μl.

### Example 2

The filtered supernatant was used to obtain topical and oral composition. The topical composition was prepared from the having ribonucleic acid (exRNA) in the range of 4-12 μg/ml encapsulated in a extracellular vesicle (obtained in example 1); phosphate buffer saline in the range of 0.4-1%, pH −7.4; sodium chloride 0.9%; and glycerin in the range of 1-5% (Composition M9).

The oral composition is prepared from the following raw materials extracellular ribonucleic acid (exRNA) in the range of 4-12 μg/ml; phosphate buffer saline in the range of 0.4-1%, pH −7.4; sodium chloride 0.9%; and ethanol in the range of 10-20% (Composition M9-1).

### Example 3

### In-Vivo Experiment in Rat Model (Psoriasis Model)

These experiment was conducted on nine female adult albino rats having the same age (4 to 8 weeks approx. weight 120 140 g). The rats were provided with standard pelleted chow diet water for drinking. All rats were maintained under standard management conditions (50-60% relative humidity, and 12 hours light dark cycles). Before the commencement of the experiment, rats were acclimatized in the laboratory conditions for 14 days. All the experimental rats were kept under constant observation during the entire period of study. The experimental protocol was approved by the (CPCSEA) committee for control supervision on experiment on animals.

Experimental rats were divided into three groups (n=3, t=9), the first group was healthy rats, second was Control (diseased induced group psoriasis) and the third group was (Treatment group-diseased induced treated with M9 composition group). Firstly, blood samples (90-100 μl) of all groups were collected from their heart via chloroform treatment. By careful handling, the fur on the rostral back of all groups was trimmed with scissor then removed with hair removal cream. Control and treatment group received a daily topical application of 65.2 mg/cm Aldara cream (5% imiquimod) on the shaved back skin (8 cm×10 cm) for 7-10 consecutive days. Normal mice (healthy rats) in diseased and treatment group were treated similarly with a normal Vaseline cream. After 5th/6th day, when shaved skin of control and treatment group showed eroded white, scaly and cracky appearance, then the blood samples (90-100 μl) were collected again in the heparin tubes. RT-PCR were performed for the assessment of mRNA expression of the cytokines like of IL-17, IL-23, IL-6, NF-KB, TNF-ALPHA, TGF-β, IFN-γ, TLR-9, responsible for psoriasis. The treatment group was topically treated with our M9 composition (Example 1) on the skin of the rats with the help of spray (each spray containing 5 μl/2 cm2 of M9 composition). After 6-8^{th} days when skin morphology started to become normal then again blood sample (100-200 μl) were collected in the heparin tubes and RT-PCR will be performed for further assessment.

### In-Vivo Experiment in Rat Model

### Real-Time Quantitative PCR

Total mRNA was extracted from whole blood after anaesthetising the rat with chloroform; the mRNA was then transcribed to cDNA. Quantitative real-time PCR was performed using Bio rad kit. The cycling conditions comprised 40 cycles at 95° C. for 15 s and 60° C. for 30 s using a single fluorescence measurement. Melting curve analysis, for which the temperature was increased from 60° C. to 95° C. at a heating rate of 0.1° C./s using a continuous fluorescence measurement, revealed a single, narrow peak of suspected fusion temperature. Rt-PCR was performed to check the expression of various inflammatory markers like cytokines over expressed in psoriasis.

### RT PCR—RNA Isolation

Firstly, 150 μl blood taken in 1.5 ml centrifuge tube. 150 μl RNA and DNA protection reagent were added. Reagents were mixed with vortex and pipetting and then 10 ul proteinase k in every sample was added, and then mixed again in the same way. Centrifuge tube was incubated at room temperature for 30-35 minutes. Now 300 μl of 2-isopropanol per sample tube was added and then vortexed. Then centrifugation was done for 1 minute in cooling centrifuge machine at 17000 rpm. Now flowthrough was discarded and the lower tube/collector tube was changed. 500 μl RNA wash buffer was added and centrifuged at 17000 rpm for 30 sec/1 minute. Flow-through was discarded and DNA wash buffer, 80 μl/sample was again added. Sample tube was incubated at room temperature for 15-20 minute and then 500 μl RNA priming buffer was added and centrifuged at 7000 rpm for 30 sec/1 min. Flowthrough was discarded and 500 μl RNA wash buffer was added. The samples were centrifuged at 17000 rpm for 30 sec/1 min was done and flow-through was discarded. Samples were then airdried in cooling centrifuge machine at 17000 rpm for 30 sec-1 min. Mini column in a new 1.5 ml centrifuge tube was transferred and 50 ml nuclease-free water (NFW) elution buffer was added. The isolated sample was incubated at −20° C. of the deep freeze and reading (260/280 ratio) was taken by BioTek Epoch 2 microplate spectrophotometer.

cDNA Preparation

All the eluted RNA sample were normalised (Volume equalisation). RT 1 μl, RT mix 4 μl, template 15 μl, and Nuclease-free water were taken in 200 μl sterile RNase free PCR tube. Pipetting was done for proper mixing and subsequently PCR tube was run on the PCR machine for 37 mins. c-DNA was prepared and further used for RT qPCR.

Gene Specific m-RNA Amplification

Primers specific for IL-17, IL-23, IL-6, NF-KB, TNF-α, TGF-β, IFN-γ, TLR-9 were used for gene specific m-RNA amplification. The m-RNA expression were assessed in real-time PCR by reading the plates BioTek Epoch 2 microplate spectrophotometer read at reading (260/280 ratio).

### Results

A number of cytokines are overexpressed in psoriasis including TNF-α, TLR-9, NF-KB, IL-6, IL-18, REL-A, TLR-8, TGF-β, IL-12 in blood samples of the imiquimod (IMQ)-induced psoriasis-like rat model. In this study, two-time points (0, 12 h) were used to test for cytokine mRNA levels. Compared with the control group, the mRNA levels of IL-17, IL-23, IL-6, NF-KB, TNF-α, TGF-β, IFN-γ, TLR-9 were significantly increased in blood samples from IMQ-treated mouse rostral back. In contrast, the mRNA levels of IL-17, IL-23, IL-6, NF-KB, TNF-α, TGF-β, IFN-γ, TLR-9 were significantly downregulated in the M9 composition-treated group when compared with the IMQ treated group.

Results showed that TNF-α, was the most significantly increased cytokine before drug were administered. The results indicate that TNF-α may play a more important role than other in IMQ-induced psoriasis like inflammation, whereas mRNA expression level in the drug-treated group is significantly alleviated.

### Example 4

### In-Vivo Experiment in Rat Model (Atopic Dermatitis Model)

### Model for Atopic Dermatitis (AD)

The rat model was established using the depilatory paste on the back skin of rats. The depilatory area was 4 cm×4 cm. 2, 4-dinitrochlorobenzene (DNCB) acetone solution from, Veet depilatory paste were purchased from Gaya, India. On the first day of experiment, 100 L of 7% DNCB acetone solution was collected with pipette and smeared evenly onto the skin area for first sensitization; the second sensitization was conducted after 10 days with 50 mL of 5% DNCB acetone solution.

Animal grouping and drug administration were assigned to three groups as disclosed in Example 2. In the Control group, rat did not receive further intervention after depilation. Based on the rat body surface area, the dosage of M9 composition (Example 1) administered orally was 1 ml/day for 10 days. Rats were administered the respective treatment three times per day for 10 consecutive days. No further intervention was performed in the or model group.

### Assessment of Itch

Rats were placed into separate plastic chambers (20 cm×30 cm×20 cm). After conducting the first and second sensitization, the scratch behaviours of the rats were recorded for 30 minutes using a video camera (HD-Apple camera). The video clips were played back and the number of scratches was counted by blind experimenters who were unaware of which rats were assigned to which treatments.

### Evaluation of Skin Lesion

Skin lesions were carefully assessed by scoring systems with modification. The skin was evaluated after every 3-4 days for 4 weeks to determine the extent and severity of lesions. The unit size for the extent of skin lesions was 0.25 cm² and the dermatitis score was calculated by the severity index, which is defined in Table 1, multiplied by the unit size.

The dorsum lesions were scored for erythema and scratching behaviour was observed for 20 minutes after each sensitization. The scores for each parameter were graded as 0 (no symptoms), 1 (mild), 2 (moderate), and 3 (severe). Erythema and scratching were assessed after each sensitization by the same investigator throughout the study.

TABLE 2
Parameters and its score for skin lesions

| PARAMETER | SCORE |
|---|---|
| No symptoms | 0 |
| Mild | 1 |
| Moderate | 2 |
| Severe | 3 |

### Collection of the Blood

After 6 weeks of behavioural tests, rats from each group were made unconscious using chloroform and the blood in the right atrium was collected using a 1 ml syringe with a 26-gauge needle. The obtained blood samples were centrifuged with 250 g in 4° C. for 10 minutes. The supernatant was collected and the procedure repeated two more time.

### Quantitative RT-PCR

RNA was extracted from blood sample according to the manufacturer's protocol. The extracted RNA samples were reverse-transcribed using reverse transcriptase according to the manufacturer's protocol. The cDNA samples were amplified by quantitative PCR according to the manufacturer's instructions.

### Results

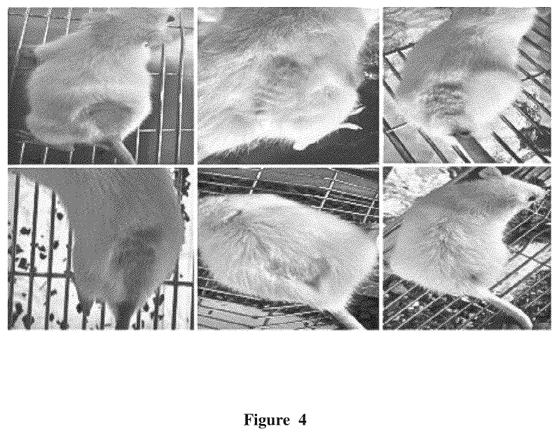

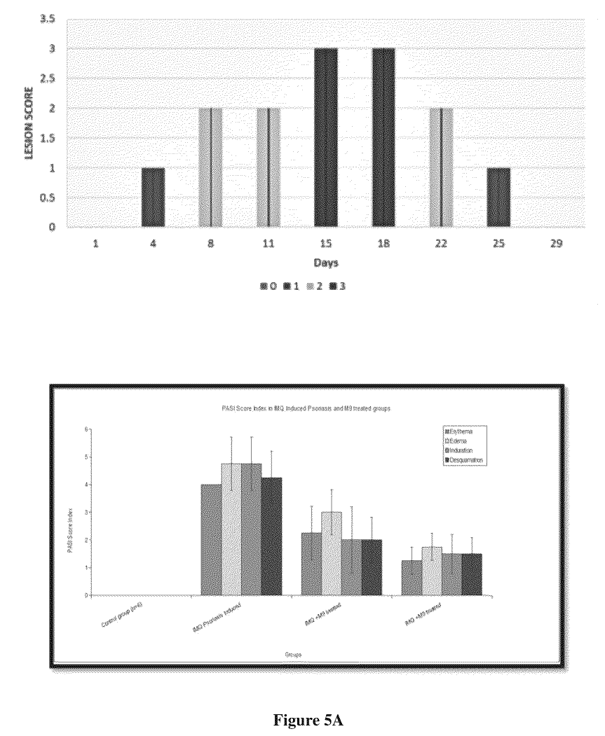

M9 composition ameliorates skin lesions in rats with AD. First, we examined the skin of animals. In rats of the healthy groups, the back skin was light red-coloured, delicate, and smooth, with evident soft texture in first photograph of FIG. 4. In rats of the model group, the back skin presented obvious lesions, including skin erythema, infiltration, scabs, and hyperpigmentation, and was scaly, rough, and thickened, thus meeting the diagnostic criteria for chronic eczema. These skin lesions improved significantly in the model rat, as shown by reduction or complete suppression of erythema, infiltration, scales, scabs, roughness, thickening, pigmentation, and scratching (FIG. 4 and Table 1). Remarkably, improvement by treatment with M9 composition were observed.

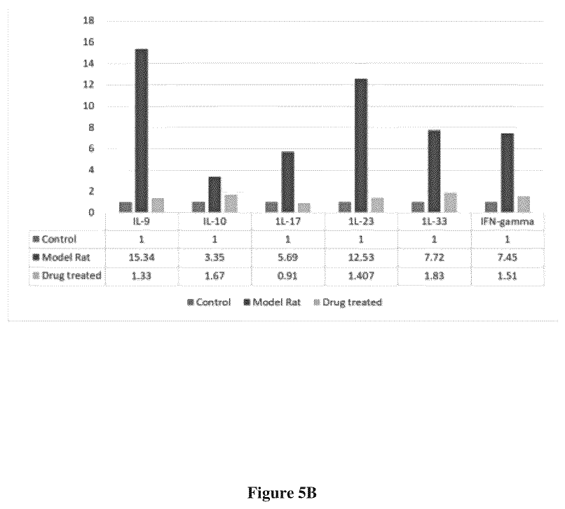

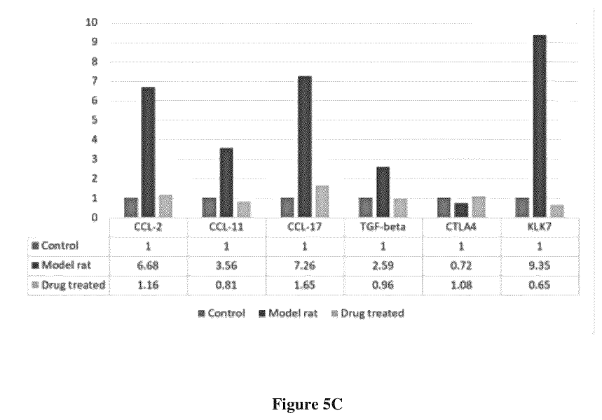

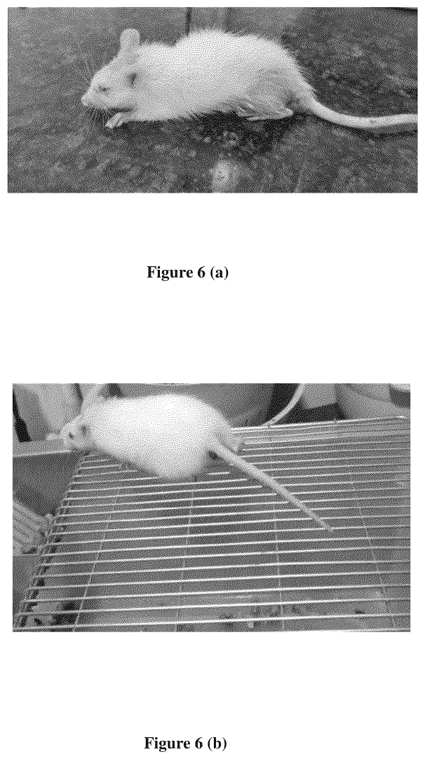

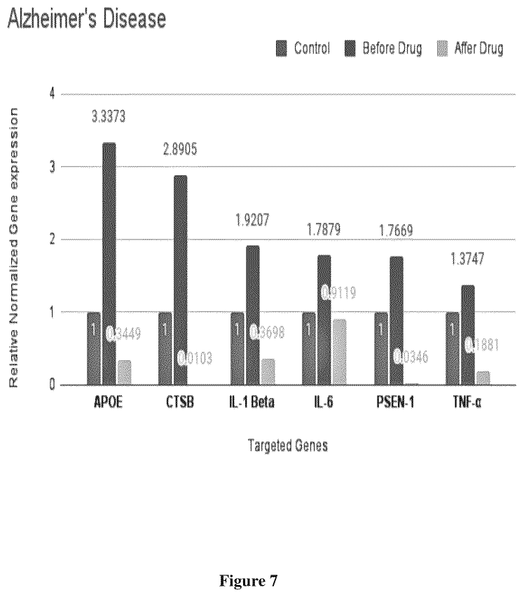

M9 composition distinctly affect serum levels of inflammatory factors in rats with chronic eczema. To elucidate the effects of M9 composition from an immune perspective, the serum of all three groups of rat for cytokines were analyzed. After conducting the sensitization, serum levels of pro-inflammatory cytokines IL-9, IL-10, IL-17, IL-23, IL-33, IFN-γ, TGF-β and CTLA4 were significantly higher in the model rat compared with the heathy rat group. Conversely, in the model rat, the serum levels of IL-9, IL-10, IL-17, IL-23, IL-33, IFN-gamma, TGF-β and CTLA4 significantly decreased after treatment with the drug in FIG. 5B.

TABLE 3
Cytokine profile in healthy rats, Control -diseased induced
group -atopic dermatitis and Treatment group- diseased
induced- treated with M9 composition group).

| | IL-9 | IL-10 | IL-17 | IL-23 | IL-33 | IFN-gamma |
|---|---|---|---|---|---|---|
| [symbol] Control | 1 | 1 | 1 | 1 | 1 | 1 |
| [symbol] Model | 1 [symbol] .34 | 3. [symbol] 5 | 5.69 | 12.53 | 7.72 | 7.45 |
| Rat | | | | | | |
| [symbol] Drug | 1.33 | 1.67 | 0.91 | 1.407 | 1.83 | 1. [symbol] 1 |
| treated | | | | | | |

[symbol]

Control [symbol]
Model Rat [symbol]
Drug treated [symbol]
indicates data missing or illegible when filed

After sensitization, serum levels of pro-inflammatory chemokines CCL-2, CCL-11 and CCL-17 significantly increased in the Control as compared to the healthy whereas the serum levels of CCL-2, CCL-11 and CCL-17 decreased after treatment with the drug. On the other hand, serine protease KLK7 significantly increased after sensitization when treated with the drug significantly decreased.

TABLE 4
Cytokine profile in healthy rats, Control -diseased
induced group -atopic dermatitis and Treatment group-
diseased induced- treated with M9 composition group

| | | | | | | |
|---|---|---|---|---|---|---|
| [symbol] Control | 1 | 1 | 1 | 1 | 1 | 1 |
| [symbol] Model | 6.68 | 3.56 | 7.26 | 2.59 | 0.72 | 9.35 |
| rat | | | | | | |
| [symbol] Drug | 1.16 | 0.81 | 1.65 | 0.96 | 1.08 | 0.65 |
| treated | | | | | | |

[symbol]

Control [symbol]
Model rat [symbol]
Drug treated

The results clearly show that M9 composition reduced the levels of inflammatory factors pro-inflammatory cytokines-chemokines IL-9, IL-10, IL-17, IL-23, IL-33, IFN-γ, CCL-2, CCL-11, CCL-17, TGF-β, CTLA4 and serine protease released by the skin cells KLK7 γ that were overexpressed in the serum of rats with chronic eczema, while decreasing the serum level of anti-inflammatory factors, thereby alleviating the inflammatory manifestations, itching, and the clinical symptoms of Atopic dermatitis. M9 composition clearly modulates the multiple gene expression program including differentiation of keratinocytes, lipid metabolism, cell cycle, and immune response.

### Example 5

### In-Vivo Experiment in Rat Model (Alzheimer's Disease Model)

### Model for Alzheimer's Disease

Study was conducted on six male adult albino rats (180-200 g) having the same age and same weight. Rats were maintained as described above. All rats were divided into 3 groups in pre experiment normal group (N1 & N2) control group (C1& C2) and treatment group (T1 & T2). The solution of colchicine induced in rat in the treatment and control group. A single dose of 15 μg or 6 μg doses of colchicine injected at 3 different sites in the striatum. Body weight of all the group were measured daily. M9-1 composition was administered 500 μl via gavage needle into the oesophagus to rat models BID for 2 weeks in the treatment group.

All rats were monitored for consecutive days to determine their physiological changes by testing Behavioral assessment-Morris water maze and open field tasks were performed. After 3 days blood of control and treatment group were collected for biochemical test include Hippocampal tissue levels of brain derived neurotrophic factor, Amyloid beta peptide test, Glutathione reductase test, Glutathione peroxidase test and blood samples were collected for Rt PCR analysis.

RNA Isolation, cDNA Preparation and RT qPCR—

RNA was isolated and cDNA was prepared as described above. cDNA 2 μl, primers 4 μl and SYBR green was added in a PCR tube and volume makeup was done with NFW. Pipetting was done for proper mixing and then each sample in triplet was loaded in 96 well plates. Plate sealer was applied and subsequently, the plate was put on the rotor for proper mixing. Now plate was transferred to the PCR machine and according to desired primers temperature was set. And PCR was run for 1 hour 52 minutes. cq value of desired genes was analyse at 80-93° C., multi curved were checked. Gene expression was analyse for given genes.

### Result

The male albino rat was introduced with colchicine treatment for the assessment of Alzheimer's disease. During this I considered three parameters for the assessment

### Morphological Changes

Following treatment of colchicine, loss in body weight and inactivity in all control group (C1& C2) and treatment group (T1 & T2) were observed as compared to before the treatment. M9-1 composition was administered 500 μl via gavage needle into the oesophagus to rat models BID for 2 weeks in the treatment group.

### Behavioural Assessment

Following test was conducted to assess the mental stability and motor neuron capacity
(I) Grip test:—measures the neurological functions maximal muscle strength of forelimbs and combined forelimbs and hindlimbs by using rope for riding. Rats able to move on the rope up to 6 min (maximum) continuously before colchicine treatment. After inducing colchicine, within 7 days rat was able to travel on rope for 1 min only (maximum). After treatment with our M9 composition for 12 days a neurological functions improved as the treated group were able to travel on rope for 3-4 min.
(II) Swimming test:—measures the neurological functions motor neuron capacity. Before treatment, rats in all groups were able to swim 2 mins (maximum) but after inducing colchicine the swim time reduced to 30-40 seconds Following treatment with M9 composition ability to swim in the treated group was reinstated for 1 min.
(III) Open field test:—Before inducing with colchicine, rats in all groups were active up to for 5 min, after inducing colchicine the rats in treated in group became significantly inactive. After treatment with M9 composition activity returned to normal (active up to for 5 min).

### Cytokine Profile

M9-1 composition downregulated the high mRNA levels of Apolipoprotein E (APOE), Cathepsin B (CTSB), IL-1 beta, IL-6, Presenilin 1 (PSEN 1) and TNF alpha in blood samples of the Colchicine induced Alzheimer's disease-like rat model. Compared with the control group, and the induced Rat the mRNA levels of APOE, CTSB, IL-1 beta, IL-6, PSEN 1 and TNF alpha were significantly increased in blood samples of colchicine induced rat. In contrast, the mRNA levels of APOE, CTSB, IL-1 beta, IL-6, PSEN 1 and TNF alpha significantly downregulated with M9 composition when compared with the Colchicine induced rat. Results showed that APOE, CTSB, IL-1 beta, PSEN 1, TNF alpha were the most significant increased biomarkers and cytokines before drug compared with other biomarkers and cytokines after drug it get downregulated IL-6 is also downregulated after drug.

### Example 6

### In-Vivo Experiment in Rat Model (Chronic Obstructive Pulmonary Disease (COPD) Disease Model)

### Model for Chronic Obstructive Pulmonary Disease

Experimental rats were maintained as described above. All rats were divided into 2 groups: PBS-group (n=3) as control and CSE group (n=3) as induced. In pre-experiment, 3 rats were injected intraperitoneally with PBS at day 0, day 11, and day 22. Simultaneously, 3 rats were injected intraperitoneally with CSE at day 0, day 11, and day 22 for pre-experiment to detect the quantitation of CSE. The LD50 in rats for nicotine given intraperitoneally was respectively 10 mg/kg. The LD50 in mice for tar was respectively 0.01 mg/g. According to LD50, 0.3 mL CSE was administered for pre-experiment. After every dosage, blood sample (100 microlitre) should collected in heparin tubes from the rats. RT-PCR will performed for the assessment of mRNA expression of cytokines and chemokines involved in COPD pathogenesis as mentioned above. At day 28, all rats will have injected intraperitoneally with chloral hydrate (3 mL/Kg) for anaesthesia, and plethysmograph was used to evaluate pulmonary function of all rats. The rats were assessed with adaptation test at a speed of 10 m/min for 10 min on a running treadmill machine.

### Preparation of CSE

At first day, 8 Cigarettes (Tar: 12 mg, Nicotine: 1.1 mg, Carbon Monoxide: 14 mg) were taken. The eight cigarettes 23 mm marked from the end with a permanent marker. A clean 1000-1 pipette tip was attached firmly inside the apparatus which was located in a fume hood. A 50-ml falcon tube were filled with 35 ml saline and attached filled tube to the apparatus. The sides of the 50-ml glass plunger were greased with petroleum jelly to ease use. An ashtray was made with foil and place at the bottom of the cigarette location to catch falling ashes. Glass syringe was attached to the small apparatus tubing, and cigarettes were inserted into the large tubing by carefully twisting and pushing; making sure that the cigarette did not break and sealed tightly to prevent smoke leakage. Cigarettes were lit, timer started, and syringe drawn up to a volume of 35 ml. Small tubing were detached from the syringe and the smoke in the syringe (i.e. depress plunger) was “exhaled” onto a paper towel. 35-ml “puffs” were repeated every 30 sec until the black mark on the cigarette were reached. The number of puffs were recorded (should be 8 to 10). Cigarette with the help of forceps removed carefully. A new cigarette was inserted and repeated until all cigarettes are smoked. Conical tube were removed from the apparatus and extract was poured into the clean, foil-wrapped 50-ml polypropylene conical tube. The pH was adjusted between 7.2 and 7.4 with 4 to 5 drops of 1 M NaOH. CSE were stored in the fridge for up to 24 hr.

### Administering Fatigue Test to the Rats.

1. Warming up the rats at a speed of 10 m/min for 5 min.
2. Increasing the speed to 15 m/min for 10 min.
3. Increasing the exercise intensity: increasing the speed by 5 m/min, starting at 20 m/min, every 30 min until rats are not able continue running.

The total running distance and running time as fatigue distance and fatigue time, respectively were recorded. Induced group were administered with M9-1 composition 500 μl via gavage needle into the oesophagus of the rat models, BID for 10 consecutive days in the treatment group.

After prescribed dosage, blood sample will have collect and RT-PCR for specific genes chemokines, cytokines which is responsible for COPD were performed for the further assessment of disease condition.

Current COPD models, such as lipopolysaccharide (LPS) or the porcine pancreatic elastase (PPE)-induced emphysema model, generate COPD-like lesions in the lungs and airways but do not otherwise resemble the pathogenesis of human COPD. A cigarette smoke (CS)-induced model remains one of the most popular because it not only simulates COPD-like lesions in the respiratory system, but it is also based on one of the main hazardous materials that causes COPD in humans. However, the time-consuming (at least 3 months) and labor-intensive aspects of the CS-induced model dramatically limit its application in new drug screening. So alternatively, to mimic the exposure of cigarette smoke (CS) we can use intraperitoneal injection of cigarette smoke extract (CSE) that can produce significant COPD like condition in mice model.

This model demonstrated the following:
1) decreased forced expiratory volume 25, 50, and 75/forced vital capacity (FEV25/FVC, FEV50/FVC, and FEV75/FVC), indicating the deterioration of lung function.
2) enlarged lung alveoli, with lung parenchymal destruction
3) reduced fatigue time and distance; and
4) increased inflammation.

Taken together, these data demonstrate that CSE model is a reliable animal model that is similar to humans because CSE exposure is the major causal factors of COPD in human. Additionally, it only took 6 weeks, to create an COPD induced model, whereas it required 3-12 months to induced the cigarette smoke model, indicating that the CSE model might be a good choice for COPD research.

### Result

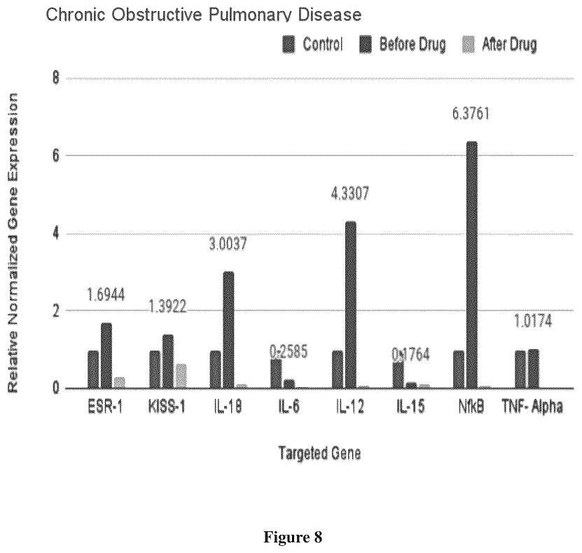

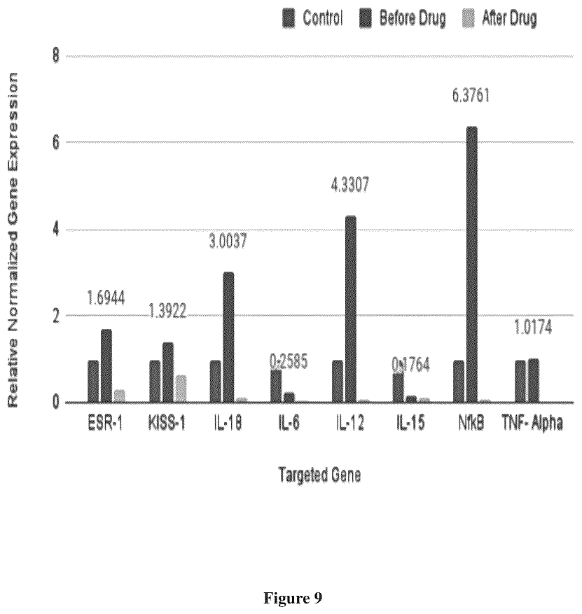

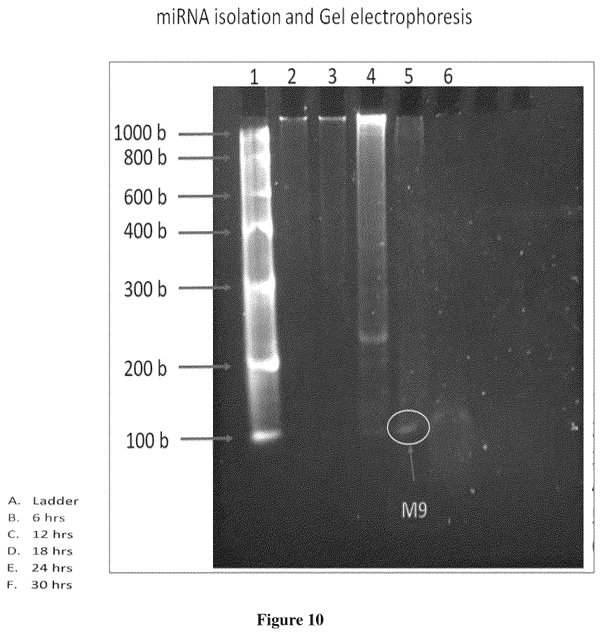

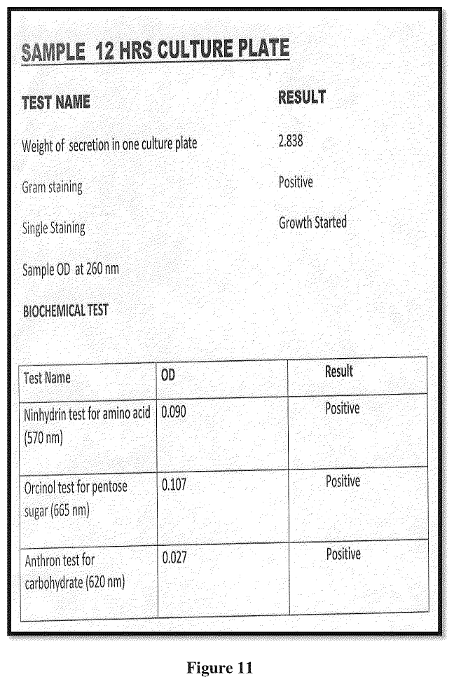

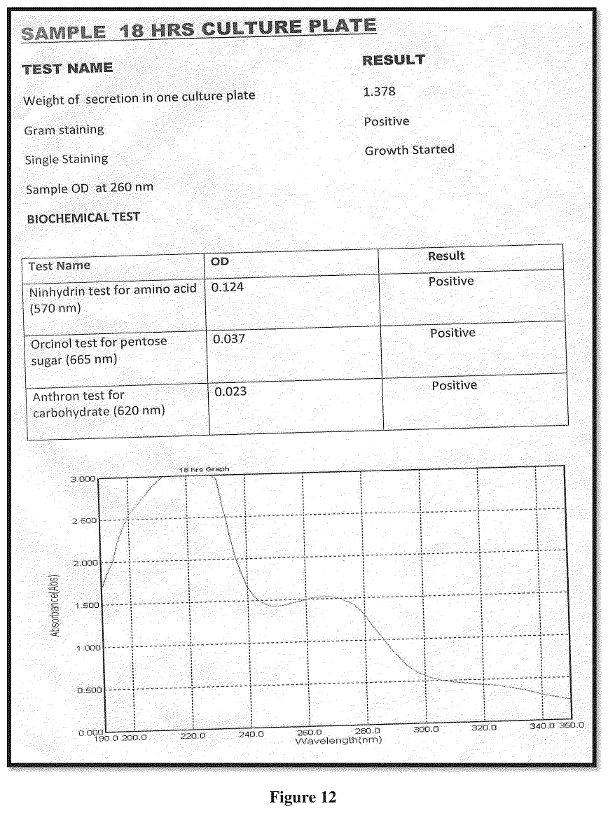

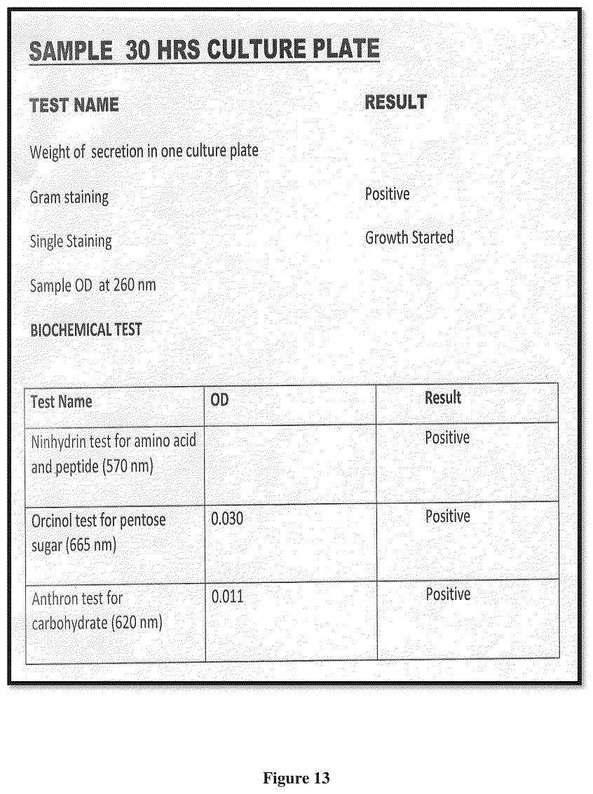

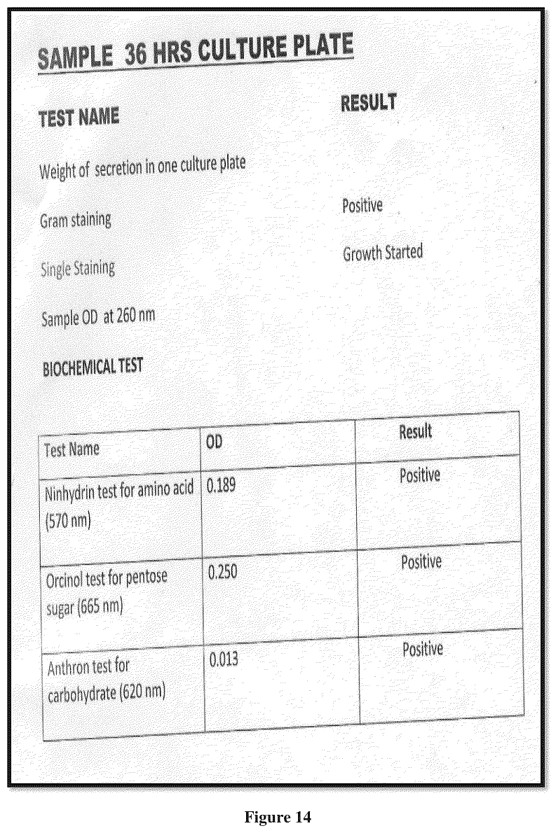

M9 composition have shown significantly reduced expression of inflammatory cytokines and various genes involved in chronic obstructive pulmonary disease. In particular the M9 composition showed significant downregulation of genes IL-17, IL-6, TGF-β, TNF-α (data shown in FIG. 8).

### Example 7

### In-Vivo Experiment in Rat Model (Polycystic Ovary Syndrome (PCOS))

### Model for Polycystic Ovary Syndrome (PCOS)

Experimental rats were maintained as described above. The study was conducted on female adult albino rats (120-150 g) having the same age and same weight. All rats were divided into 3 groups in pre experiment normal group were injected with 200 ul sesame oil, control and treatment group were injected with 0.006 g DHEA+200 ul Sesame oil solution. This solution was induced continuously for 21 days to the treatment and control group. Body weight of all group measured daily. All rats were monitored for consecutive days to determine their physiological changes. After 21 days blood of control and treatment group were collected for Rt-PCR analysis. The treatment group treated M9-1 composition, 3 ml orally seven consecutive days. After treatment for 7 days with M9 composition in treatment group then again blood were collected for RT analysis.

All PCOD specific genes including Estrogen Receptor 1 (ESR-1), KISS-1 and inflammatory cytokines including IL-18, IL-6, IL-12, IL-15, NF-KB, Insulin receptor substrate-1 (IRS-1), Tumor necrosis factor-alpha (TNF-α), C-X-C motif chemokine-10 (CXCL-10) were downregulated in RT-PCR analysis (FIG. 8).

While specific language has been used to describe the disclosure, any limitations arising on account of the same are not intended. As would be apparent to a person in the art, various working modifications may be made to the method in order to implement the inventive concept as taught herein. The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

1-22. (canceled)

23. An anti-inflammatory composition that downregulates inflammatory genes comprising;
a) extracellular ribonucleic acid (exRNA);
b) extracellular vesicle bound by a single membrane phospholipid layer comprising glycopeptides; lipopeptide; polysaccharides; short chain fatty acids including propionic acid and small peptides;
c) phosphate buffer saline;
d) sodium chloride;
e) glycerin; and
f) double distilled water
wherein the extracellular ribonucleic acid (exRNA) is encapsulated in an extracellular vesicle (EV),
wherein the extracellular ribonucleic acid (exRNA) and the extracellular vesicle (EV) are derived from an extra polymeric substances (EPS), and
wherein the extra polymeric substances (EPS) are derived is from gram positive non-pathogenic bacteria Bacillus velezensis MTCC25513.

24. The composition as claimed in claim 23, wherein the extracellular ribonucleic acid (exRNA is in the range of 4-12 g/ml; the phosphate buffer saline is in the range of 0.4-1%, pH −7.4; the sodium chloride is at the concentration 0.9%; and glycerin is in the range of 1-5%.

25. The composition as claimed in claim 23, wherein the extracellular vesicle is anionic molecule with a zeta potential (−1) to (−30) mV and is 20-500 nm in size.

26. The composition as claimed in claim 23, wherein the extracellular ribonucleic acid (exRNA) is selected from a group comprising micro Ribonucleic acid (miRNA), small interfering Ribonucleic acid (siRNA), guide Ribonucleic acid (gRNA) cRNA (circular Ribonucleic acid), piwi Ribonucleic acid (piRNA).

27. The composition as claimed in claim 23, wherein the composition is administered topically.

28. An anti-inflammatory composition that downregulates inflammatory genes comprising;
a) extracellular ribonucleic acid (exRNA);
b) extracellular vesicle bound by a single membrane phospholipid layer comprising glycopeptides; lipopeptide; polysaccharides; short chain fatty acids including propionic acid; and small peptides;
c) phosphate buffer saline;
d) ethanol;
e) sodium chloride; and
f) double distilled water,
wherein the extracellular ribonucleic acid (exRNA) is encapsulated in an extracellular vesicle (EV),
wherein the extracellular ribonucleic acid (exRNA) and the extracellular vesicle (EV) are derived from an extra polymeric substances (EPS), and
wherein the extra polymeric substances (EPS) is derived is from gram positive non-pathogenic bacteria Bacillus velezensis MTCC25513.

29. The anti-inflammatory composition as claimed in claim 23 that downregulates the pro-inflammatory gene selected from a group comprising APOE (Apolipoprotein E), CTSB (Cathepsin-B), IL-1β (Interleukin-1 beta), IL-6 (Interleukin-6), Presenilin 1 (PSEN-1), TNF-α (Tumour necrosis factor-α), IL-4 (Interleukin-4), IL-5 (Interleukin-5), IL-6 (Interleukim-6), IL-9 (Interleukim-9), IL-12 (Interleukin-12), IL-13 (Interleukin-13), IL-15 (Interleukin-15), IL-17 (Interleukin-17), IL-18 (Interleukin-18), IL-23 (Interleukim-23), IL-33 (Interleukim-33) (Interleukim-6), TGF-β (Transforming growth factor-β), TNF-α (Tumour necrosis factor-α), IL-17, IL-23, IL-6, NfκB (Nuclear Factor kappa-light-chain-enhancer of activated B cells), TLR-9 (Toll like receptor-9), TNF-α, TGF-β, IFN-γ (Interferon-γ), IRS-1 (Insulin receptor substrate 1), CXCL-10 (C-X-C motif chemokine ligand 10), ESR-1 (Estrogen Receptor 1), KISS-1, KLK-7 (Kallikrein-1), CCL-II (C-C motif chemokine ligand-11), CCL-2 (C-C motif chemokine ligand-2).

30. The composition as claimed in claim 28, wherein the composition is administered orally.

31. The composition as claimed in claim 28, wherein the extracellular ribonucleic acid (exRNA) in the range of 4-12 μg/ml; the phosphate buffer saline in the range of 0.4-1%, pH −7.4; the sodium chloride is at 0.9%; and the ethanol in the range of 10-20%.

32. A method of treating autoimmune diseases including psoriasis, Polycystic ovarian disorder (PCOD), and Coronary obstructive pulmonary disorder (COPD), asthma, Alzheimer's diseases, and atopic dermatitis with a therapeutically effective amount of the composition as claimed in claim 28.

33. A method of conversion of biofilm-associated extra polymeric substances (EPS) into vesicular compartments (extracellular vesicles) comprising:
a) suspension of the extra polymeric (EPS) substances in conversion buffer to obtain EPS suspension;
b) homogenization of the EPS suspension followed by the centrifugation of the homogenized EPS suspension to obtain an EPS supernatant; and
c) filtration of the EPS supernatant to obtain extracellular vesicles
wherein the extracellular vesicles encapsulate extracellular ribonucleic acid (exRNA),
wherein the suspension is made is 1:20 in the conversion buffer, and
wherein extra polymeric substances (EPS) is derived is from gram positive non-pathogenic bacteria Bacillus velezensis MTCC25513.

34. The method as claimed in claim 33, wherein conversion buffer is selected from a group comprising double distilled water; ethanol in the range of 0-30% in double distilled water; Dimethyl sulfoxide (DMSO) in the range of 0-5% in double distilled water; Sodium chloride (NaCl) (0.9% in double distilled water); Phosphate buffer saline (PBS) in the range of 0.4-1%, pH 7.4.

35. The method as claimed in claim 33, wherein the homogenization is carried out at 1000 rpm for 1-5 minutes.

36. The method as claimed in claim 33, wherein the centrifugation is carried out at 4500 rpm for 2-10 minutes.

37. The method as claimed in claim 33, wherein the filtration is carried out twice with a membrane filter pore size, of 0.45 μm followed by a membrane filter pore size, of 0.22 μm.

38. A method of treating autoimmune diseases including psoriasis, PCOD, COPD, asthma, Alzheimer's diseases, and atopic dermatitis with a therapeutically effective amount of the composition as claimed in claim 23.

39. A kit comprising the composition as claimed in claim 23 with means for reconstitution of said composition for a ready-to-use formulation.

40. A kit comprising the composition as claimed in claim 28 with means for reconstitution of said composition for a ready-to-use formulation.

41. A postbiotic composition comprising the anti-inflammatory composition as claimed in claim 23.

42. A postbiotic composition comprising the anti-inflammatory composition as claimed in claim 28.
